(19) United States
(12) Patent Application Publication
(10) Pub. No.: US 2019/0274560 A1
(43) Pub. Date: Sep. 12, 2019

(54) SYSTEM AND METHOD FOR MONITORING PERIODIC SIGNALS

(71) Applicant: CONTINUSE BIOMETRICS LTD., Tel Aviv (IL)

(72) Inventors: Zeev ZALEVSKY, Rosh HaAyin (IL); Zeev MARKMAN, Bet-Dagan (IL)

(21) Appl. No.: 16/347,951

(22) PCT Filed: Nov. 7, 2017
(86) PCT No.: PCT/IL2017/051210

§ 371 Date: May 7, 2019

Related U.S. Application Data
(60) Provisional application No. 62/420,209, filed on Nov. 10, 2016.

(51) Int. Cl. A61B 5/024; G01H 9/00; A61B 5/00

(52) U.S. Cl. CPC A61B 5/02416; A61B 5/0261; A61B 5/0077; G01H 9/00

(57) ABSTRACT

A method and system for use in detection of a periodic signal are described. The method comprising: providing data about periodicity of the periodic signal; sampling said periodic signal at a sampling rate along a plurality of periods of said signal, said sampling rate being selected as having temporal location of at least one sampling event along a corresponding period thereof varies for each period for a desired number of the periods; arranging sampled data pieces in accordance with sample time thereof with respect to time within a period of said periodic signal; and reconstructing a profile of said periodic signal from said plurality of periods being sampled at a desired resolution being greater than resolution associated with said sampling rate. The variation in temporal location of sampling event may be provided by proper selection of sampling rate, or by varying time of sampling rate in accordance with the period of the periodic signal.

### TECHNOLOGICAL FIELD

The present invention is in the field of optical monitoring of a sample, such as biological tissue, or parameters of a patient, and particularly relates to monitoring of periodic parameters with reduced sampling rate.

### BACKGROUND

Accuracy of measurement may often relay, at least in part, on measurement resolution. Reconstruction of a temporally varying signal may be provided if the signal is measured at a sampling frequency that is no lower than the Nyquist frequency, or more specifically, no lower than twice the maximal frequency of the signal.

Various techniques have been described, for use in monitoring parameters of samples such as objects, biological tissue as well as parameters of a human body (such as heart rate etc.) For example, the following patent publications describe the concept of speckle-based optical monitoring techniques.

U.S. Pat. No. 8,638,991 presents a method for imaging an object. The method comprises imaging a coherent speckle pattern propagating from an object, using an imaging system being focused on a plane displaced from the object.

US 2013/0144137 and US 2014/0148658 present a system and method for use in monitoring one or more conditions of a subject's body. The system includes a control unit which includes an input port for receiving image data, a memory utility, and a processor utility. The image data is indicative of data measured by a pixel detector array and is in the form of a sequence of speckle patterns generated by a portion of the subject's body in response to illumination thereof by coherent light according to a certain sampling time pattern. The memory utility stores one or more predetermined models, the model comprising data indicative of a relation between one or more measurable parameters and one or more conditions of the subject's body. The processor utility is configured and operable for processing the image data to determine one or more corresponding body conditions; and generating output data indicative of the corresponding body conditions.

### GENERAL DESCRIPTION

There is a need in the art for a technique enabling monitoring of signal profile at greater temporal resolution. The existing optical monitoring techniques and in particular speckle-based monitoring techniques are typically limited by sampling frequency of light collection, e.g. sampling rate of a camera unit or imager. This requires the use of expensive fast camera (as well as high intensity illumination) for signal detection with high temporal resolution, or detection of the signal with lower temporal resolution using simple and slow camera.

The technique of the present invention utilizes data about the periodicity of a signal for determining a high resolution profile thereof. This is based on data collection at a relatively lower sampling rate selected in accordance with given data on signal periodicity. To this end the present technique utilizes sampling at a selected rate such that for each period of the signal, the sample instances are temporally shifted with respect to previous period. This can be achieved by utilizing a delay in sampling between periods and/or by selecting the sampling rate such that there is no integer common denominator between the periodicity of the signal and the sampling rate. To reconstruct the profile of the periodic signal, the collected data is rearranged in accordance with relative location of each sample with respect to period of the signal. Thus, samples from a plurality of periods are combined together to reconstruct the profile of the signal at increased temporal resolution. This technique allows high resolution reconstruction of a periodic signal utilizing collection instruments having sampling rate that is much lower than the desired and achieved resolution.

It should be noted that the present technique may typically utilize illumination pulses providing sub-period sampling of the signal. More specifically, the collection unit may operate at a predetermined (relatively low) sampling rate, while the illumination unit provides short pulses that are synchronized with the period of the signal to provide sub-period sampling. Thus, the collection unit may utilize exposure time (integration time) that is longer than the pulse duration of the illumination unit, while the timing of the illumination pulses determines timing of sampling. Each illumination pulse therefore corresponds to a respective sampling event and duration and/or profile of the illumination pulse determine corresponding sampling exposure time and profile.

For example, according to some embodiments, the technique may be utilized in combination with speckle based monitoring of a sample. Generally, an inspection region of the sample is illuminated by coherent illumination of a selected wavelength range, and light returning from the sample, by reflection and/or scattering, is collected by imaging of an intermediate plane onto a detector array (e.g. using a defocused camera unit). The so-generated image data provides data about secondary speckle patterns that are the result of interference between light components returning from the inspection region. A sequence of image data pieces associated with speckle patterns provides data on the inspection region (e.g. vibrations, movement and/or changes in orientation) and by determining correlation between consecutive speckle patterns this data may be determined at a desired sampling rate corresponding to rate of collection of the image data pieces. Thus, the technique of the present invention may provide for increasing effective sampling rate utilizing data about signal periodicity and utilizing sub sampling synchronization of data collection.

In the context of speckle-based detection, the technique of the present invention utilizes sampling rate that is determined in accordance with an illumination sequence pattern. More specifically, the inspection region may be illuminated with a sequence of pulses of coherent illumination (e.g. using switched or pulsed laser). Additionally, light returning from the inspection region is collected at a corresponding sampling rate to determine a plurality of image data pieces indicative of secondary speckle patterns in the returning light along a sampling time of several periods (e.g. two or more, three or more, or higher number of periods). The plurality of image data pieces are arranged in accordance with temporal sampling location with respect to period of the signal to form a sequence of image data pieces mapping one or more reconstructed complete periods of the periodic signal. In accordance with the speckle based detection technique, the rearranged image data pieces may be further processed for determining correlation between consecutive speckle patterns (in the rearranged order) and thus for determining various desired parameters of the sample, include signal profile, at temporal resolution higher than the sampling rate.

It should be noted that the periodicity of the signal to be detected may be inherent to the signal (e.g. heat rate and blood flow profile) or induced by an external stimulation. More specifically, the system of the invention may also comprise a stimulation unit configured for applying selected periodic stimulation onto the sample, i.e. directly onto the inspection region or generally on one or more selected regions of the sample, and the system may collect data about response of the inspection region at a selected sampling rate to thereby reconstruct a response function of the sample as desired resolution (being greater than the sampling rate).

Thus according to a broad aspect of the present invention, there is provided a method for use in detection of a periodic signal, the method comprising: providing data about periodicity of the periodic signal; sampling said periodic signal at a sampling rate along a plurality of periods of said signal, said sampling rate being selected as having non-integer number of samples within a period of said periodic signal; arranging sampled data pieces in accordance with sample time thereof with respect to time within a period of said periodic signal; and reconstructing a profile of said periodic signal from said plurality of periods being sampled at a desired resolution being greater than resolution associated with said sampling rate.

Alternatively or additionally, according to a broad aspect of the invention there is provided a method for use in detection of a periodic signal, the method comprising:

providing data about periodicity of the periodic signal;

sampling said periodic signal at a sampling rate along a plurality of periods of said signal, said sampling rate being selected as having temporal location of at least one sampling event along a corresponding period thereof varies for each period for a desired number of the periods;

arranging sampled data pieces in accordance with sample time thereof with respect to time within a period of said periodic signal; and

reconstructing a profile of said periodic signal from said plurality of periods being sampled at a desired resolution being greater than resolution associated with said sampling rate.

In some embodiments, the sampling rate may be selected as having non-integer number of sampling events within a period of said periodic signal. In some other embodiments, the sampling events may be temporally shifted between periods of said periodic signal.

Sampling said periodic signals may comprise providing pulsed illumination having said selected sampling rate at an inspection region, and collecting data about light returning from said inspection region at a predetermined frame-rate, integration time of light collection being longer than pulse duration of said pulsed illumination.

Generally, in some embodiments, the sampling rate of the pulsed illumination may be determined such that each frame of the light collection corresponds to a single respective pulse of illumination.

According to one other broad aspect of the invention, there is provided a method for use in detection of periodic signal, the method comprising: providing data about periodicity of the signal, directing pulsed optical illumination of a selected repetition rate onto a region of interest for detection of said periodic signals, collecting a plurality of data pieces corresponding to speckle patterns formed in light returning from said inspection region and processing said plurality of data pieces to determine a profile of said periodic signal; wherein said processing comprises arranging said plurality of data pieces in accordance with temporal location of each data piece with respect to period of said periodic signal and determining data about correlation between consecutive speckle patterns in the arranged order, said correlation data being indicative of profile of said periodic signal.

According to some embodiments, collection of a plurality of data pieces may comprise operating a collection unit at a predetermined frame rate and integration time being longer with respect to pulse duration of said pulsed optical illumination, thereby repetition rate and timing of said pulsed illumination determines sampling instances of said signal.

Said repetition rate of the pulsed illumination may be selected in accordance with said periodicity of the periodic signals such that illumination pulses are temporally shifted between periods of said periodic signal.

The collection of a plurality of data pieces may use a sampling rate corresponding to repetition rate of the pulsed illumination.

Said pulsed illumination may generally comprise coherent illumination having a selected wavelength range.

According to some embodiments, said providing data about the periodicity of the signal may comprise stimulating the region of interest according to a predetermined periodic time profile, the periodicity of the signal being a dependent on the predetermined periodic time profile.

According to yet another broad aspect of the invention, there is provided a system for use in monitoring of a signal, the system comprising: an illumination unit configured for directing coherent pulsed illumination with a selected repetition rate to an inspection region, a collection unit configured for collecting image data associated with speckle patterns generated by light returning from said inspection region and a control unit, the control unit is configured and operable for controlling operation of said illumination unit and said collection unit and for receiving from the collection unit input data corresponding with a plurality of image data pieces of said collected data about speckle patterns and for processing said input data for determining profile of one or more periodic signals; said control unit is configured for processing said input data by arranging said plurality of image data pieces in accordance with temporal location of each data piece with respect to period of said periodic signal and determining data about correlation between consecutive speckle patterns in the arranged order.

The control unit may be configured and operable for operating the collection unit at a predetermined frame rate and integration time, and for synchronizing pulse timing of the illumination unit to provide sub-period sampling of said periodic signal.

In some embodiments, each frame captured by the light collection unit may correspond to, or be associated with, a single respective pulse of illumination by the illumination unit.

Additionally or alternatively, the control unit may be configured for determining profile of said one or more periodic signal at a resolution greater than sampling rate of any of the illumination unit and collection unit.

Further, according to some embodiments, the collection unit may comprise a camera unit configured for collecting defocused image data associated with light returning from the inspection region.

The above described system may be configured for sub-period sampling of said periodic signal, said control unit being configured for synchronizing pulse timing of said illumination unit to provide temporal shifts between illumination timing of different periods of said periodic signal.

According to some embodiments, said control unit is configured for selecting pulse timing as having non-integer number of pulses within a period of said periodic signal.

Further, according to some embodiments, the control unit may be configured for causing the illumination unit to emit a predetermined number of pulses during each period of said periodic signal and to temporally shift the predetermined number of signals between periods of said periodic signal.

The system as described herein may further comprise a stimulation unit configured for stimulating the inspection region according to a periodic time profile. In the some embodiments, the control unit may be configured for controlling an operation of said stimulation unit.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 exemplifies a system for use in remote monitoring of a sample according to some embodiments of the invention;

FIG. 2 exemplifies a configuration of a control unit for use in a system according to some embodiments of the invention;

FIG. 3 exemplifies a measuring technique according to some embodiments of the invention in a way of a block diagram;

FIG. 4 illustrates a sampling train and an exemplary periodic signal showing measuring according to some embodiments of the invention using selection of sampling rate; and

FIG. 5 illustrates a shifted sampling train and an exemplary periodic signal showing measuring according to some embodiments of the invention.

### DETAILED DESCRIPTION OF EMBODIMENTS

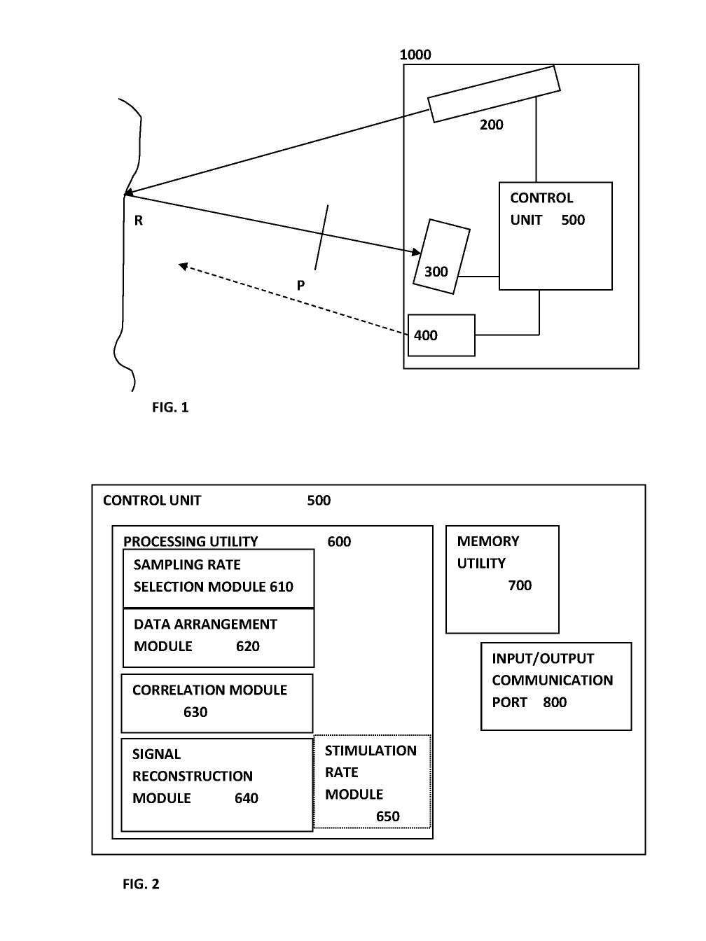

As indicated above, the technique of the present invention provides for reconstruction of a periodic signal profile at desired resolution, which may be greater than sampling rate used for measurement of the signal. Reference is made to FIG. 1 illustrating a system 1000 for use in optical monitoring of a sample in accordance with the technique of the invention. The system 1000 in this example is configured for use in speckle-based monitoring and includes an illumination unit 200 configured for directing pulsed coherent optical illumination onto an inspection region R and a collection unit 300 configured for collecting light returning from the inspection region R and generating sequence of image data pieces indicative of a secondary speckle pattern by imaging an intermediate plane P located between the inspection region R and the collection unit 300. It should however be noted that the present technique is not limited to speckle-based monitoring or optical monitoring and may be used for reconstructions of periodic signals collected in any known monitoring technique having certain sampling rate. In the present, non-limiting example of speckle-based optical monitoring, the illumination unit 200 and collection unit 300 are connected to and operable by a control unit 500 configured for determining operation mode and may also be configured for receiving and processing collected data. It should however be noted that in some embodiments, the control unit 500 may store and/or transfer the collected data for remote processing as the case may be. As also shown in FIG. 1, the system 1000 may include a stimulation module 400 configured for generating selected stimulation (e.g. ultra sound stimulation) at a selected repetition rate (stimulation periodicity/periodic time profile) and direct it onto the sample or its vicinity.

Generally the illumination unit 200 includes a light source unit, such as a laser unit (e.g. q-switch or otherwise pulsed laser), and may include one or more optical elements for properly directing and shaping the generated illumination towards the inspection region R to form one or more illumination spots on the inspection region R. In some configurations the optical arrangement may include a controllable shutter configured for modulating generated illumination (e.g. when continuous wave (CW) light source is used) and providing pulsating illumination at a selected repetition rate and pulse duration, alternatively Q-switch laser or any other pulsed laser may be used. Repetition rate of light provided by the illumination unit 200 is controllable by the control unit 500 and typically selected in accordance with known or pre-provided data about periodicity of the periodic signal to be measured. The control unit may also enable control on pulse duration enabling to optimize sampling of the periodic signal.

The collection unit 300 generally includes an optical arrangement and a detector array configured to collect image data based on an intermediate plane P located between the inspection region R and the detector array. More specifically, the optical arrangement and the detector array are arranged, by relative location between them and optical power of the optical arrangement, for imaging an intermediate object plane, and thus providing defocused imaging of the inspection region R. This forms image data corresponding with a secondary speckle pattern generated due to interference of light components returning from the inspection region by reflection and/or scattering. The collection unit 300 is typically operable by the control unit 500 at a selected sampling rate and optionally exposure time. The sampling rate is selected in accordance with repetition rate of the illumination unit such that for each illumination pulse, a corresponding image data piece can be collected. The exposure time may be selected to be sufficiently short to allow sampling at the selected rate, however the effective exposure time may be determined by pulse length of the illumination unit 200 allowing the use of relatively slow camera unit or detector.

The control unit 500 is configured and operable for controlling operation of the illumination unit 200 and collection unit 300 and, in some embodiments, of the stimulation unit 400. The control unit 500 may generally be configured for receiving and processing collected image data pieces in accordance with the technique of the invention, or for storing and transmitting the collected image data pieces for further processing in according with some embodiments of the invention. FIG. 2 illustrated schematically a configuration of the control unit 500 according to some embodiments in more details. In this connection the control unit 500 may be configured as a computing unit including one or more processors, memory utility, input/output communication ports and/or control interface. The control unit 500 may assign memory sectors for installing input or pre-stored data about periodicity of the signal to be detected and utilize this stored data for controlling operation of the illumination and collection unit (200 and 300) in accordance with the present technique and for processing input data received from the collection unit via input port to provide data about the periodic signal at selected temporal resolution.

As shown in FIG. 2, the control unit may typically include a processing utility 600, memory utility 700 and input/output communication port 800 generally configured for transmitting and receiving data between the control unit 500 and the illumination 200, collection 300 and/or stimulation 400 units, as well as to provide network communication and user interface when used.

The processing utility 600 may generally be implemented as one or more computer processors and include several hardware and/or software modules associated with an operation thereof. Additionally the processing utility includes a sampling rate selection module 610 configured for selecting the sampling rate of the system (e.g. repetition rate of the illumination unit 200 and optionally corresponding sampling rate of the collection unit 300); data arrangement module 620 configured for receiving collected input data and arranging the received data in accordance with relative time stamp and period of the periodic signal as will be described in more details further below; correlation module 630 configured for receiving a sequence of input data pieces and determining correlation between consecutive data pieces; and signal reconstruction module 640 configured for determining a profile of the reconstructed signal in accordance with correlation determined by the correlation module 630. The processing utility 600 may also include a stimulation rate module 650 configured for controlling rate/periodicity of external stimulation applied to the sample when used.

The sampling rate selection module 610 is configured and operable for determining rate of operation of the illumination unit 200 in accordance with known or pre-provided data about periodicity of the periodic signal to be measured. In some configurations, such data on periodicity of the periodic signal to be measure may be determined and stored in the memory utility in initial measurement performed by the system. More specifically, to enable processing of collected data for reconstruction of the measured signal with desired resolution, different periods of the signal are preferably sampled at different time locations within each period. To provide such shift in sampling instances between periods, the sampling rate selection module 610 may determine and control the illumination and/or collection units (in the present example the sampling rate may be determined by repetition rate and temporal location of the pulsating illumination) sampling data from the inspection region using one or more of the following techniques: sampling rate of the system may be selected in to have minimal common denominator with a periodicity of the periodic signal that is higher than selected threshold, e.g. higher than 3, 4, 5 or any other selected threshold; sampling instances may be shifted between periods to provide shifted sampling instances.

It should be noted that the sampling rate and synchronization between timing of sample (speckle pattern) collection with respect to signal period may generally be selected irrespective of actual sampling rate of the collection unit 300. More specifically, the collection unit 300 may be operated for collecting illumination and generating corresponding image data pieces at a predetermined frame rate (in accordance with unit capabilities), while the sub-period synchronization is determined by timing of the illumination pulses of the illumination unit 200. This provides for sub-period sampling by adjustment of the illumination timing (illumination sequence) with a generally predetermined collection timing (frame rate). As mentioned above, the illumination timing and the collection timing are preferably selected such each frame only collects light from one illumination pulse. Since the timing of the illumination pulse is known, the data arrangement module 620 is configured for arranging the received data in accordance with relative time stamp according to the timing of the light pulses.

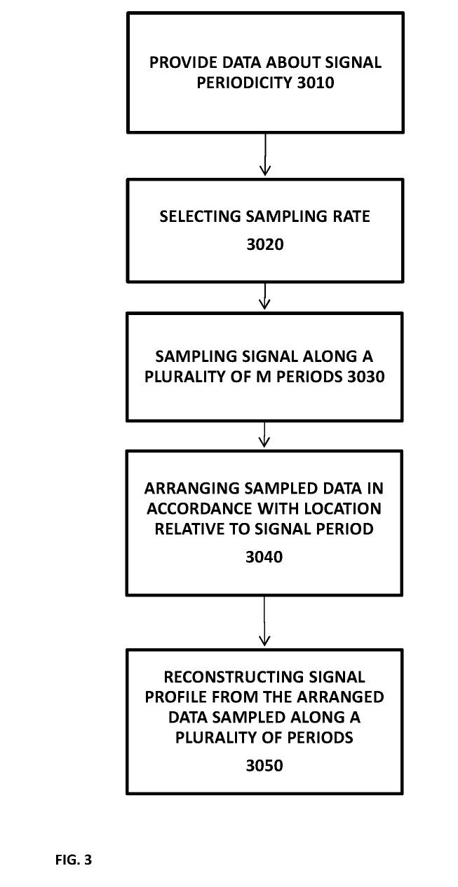

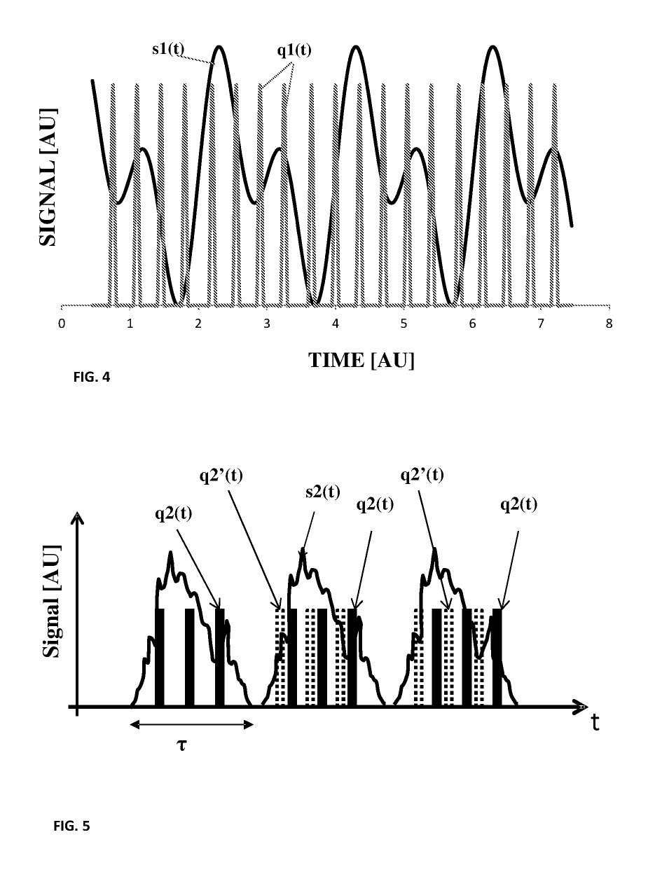

Therefore, generally the system may be provided with data about periodicity of a signal to be detected, this data may be determined in any known technique by the system or by an external system. For example, the system 1000 may be initially operated in a periodicity detection mode for monitoring the inspection region R and determining periodicity of the signal to be detected utilizing speckle-based monitoring as described e.g. in U.S. Pat. No. 8,638,991. In accordance with data about periodicity of the periodic signal to be detected, the sampling rate selection module 610 may operate to determine preferred selected sampling frequency for measuring of the corresponding periodic signal. The sampling rate may be selected such that the sampling rate and periodicity of the periodic signal have relatively high minimal common denominator (e.g. greater than a predetermined threshold relating to number of periods to be folded by re-arrangement of the sampled data), and thus temporal variations between the signal period and the sampling instances change with time and certain number of cycles are required for the signal and the sampling to complete cycles together. For example, when measuring a periodic signal having periodicity of 60 events per minute (e.g. heart rate at a rate of one pulse per second or 1 Hz) the sampling rate selection module 610 may determine a non-integer sampling rate of e.g. 5.2 Hz, 3.7 Hz 10.2 Hz etc. Thus within each second, the temporal location of the sampling instances changes (with respect to period of the periodic signal or to any given clock). Alternatively, the sampling rate selection module 610 may operate for selecting a predetermined sampling rate regardless of periodicity of the periodic signal to be detected, and introduce selected or random temporal shifts between sampling instances to generate shifts between sampling instances and period of the measured signal. Examples of these two sampling rate relations are illustrated in FIGS. 3 and 4 and will be described in more details further below.

The sampling rate selection module 610 is further configured and operable for operating the illumination unit 200, and in some embodiments also the collection unit 300 or generally any other unit the system 1000 may be equipped with for collection of sample data pieces, for sampling data about the periodic signal at the selected rate. In this example of speckle based monitoring, the illumination unit 200 is generally operated to provide pulsed coherent illumination with pulse length that is relatively short (as compared to the selected repetition rate) such that the temporal location of illumination pulses determine the sampling instances. The collection unit 300 may be operated at a predetermined sampling rate, while being configured with image acquisition time (integration time) longer than duration of the illumination pulses, such that the sampling is actually determined by the pulsating illumination. Alternatively, in some embodiments, the collection/sampling unit may also be synchronized with the desired sub-period sampling. As indicated above for speckle-based monitoring, the collection unit may generally collect image data associated with an intermediate plane along light propagation between the inspection region R and the collection unit 300 to thereby generate image data pieces associated with secondary speckle patterns. Thus the collection unit generates a data sequence including a plurality of data pieces associated with the corresponding sampling instances.

The data sequence collected by the collection unit (or by any other type of sampling unit if used) is transmitted to the control unit 500 for rearrangement and processing, generally via the input communication port 800 or a sub-port thereof. Generally, the data arrangement module 620 of the processing utility 600 is operable for receiving the sequence of the sampled data pieces from the collection unit 300 and for determining arrangement of the sampling instances (data pieces) in accordance with time shifts thereof with respect to period of the signal to be measured. The data arrangement module 620 may be configured and operable to determine for each data piece associated with a sampling instance, a relation between time stamp of the data piece and from a selected reference point of the periodic signal (e.g. time from period start). For example, the data arrangement module may determine data indicative of time of sampling instance (time stamp) modulo the period length of the periodic signal, and arrange the collected data pieces in accordance with the determined modulo operation. Thus, the collected sampling data pieces are arranged in accordance with their relative location along a few (one, two or three) periods of the periodic signal allowing for high resolution reconstruction of the signal. The reconstructed resolution is determined based on number of sampling instances in the reconstructed signal, which relates to the number periods of the signal used in the sampling and reconstruction.

Generally in some configurations, the desired signals may be reconstructed with over sampling, i.e. two or more periods are reconstructed, or two or more sampling instances are used for each time stamp. This is used to provide averaging of the measured signals and smooth out variations between repeating periods and/or noise.

For example, considering one period of the periodic signal as s(t), and the sampling frequency being selected as 1/δt such that the sampling repeats itself after M periods (i.e. effectively increasing sampling bandwidth by a factor of M). Thus, data collected from M periods (e.g. M=3, 5 or any desired number) enables corresponding increase in reconstruction resolution. Alternatively, the data sampled from M periods may be rearranged to form two or three periods of the signal, providing sampling resolution increase by M/2 or M/3 while averaging the two or more signal periods for decreasing effects of noise.

The sampling is done by illuminating the sample by pulsed laser (illumination unit 200). By properly adjusting the frequency and/or temporal position of the sampling pulses of the laser the following 3 set of samples is obtained:

$$s ( t ) \cdot {\sum }_{n} δ ( t - n δ t ) = {\sum }_{n} s ( n δ t ) δ ( t - n δ t ) \qquad ( equation 1 )$$

for the first period of the measured periodic signal;

$$s ( t ) \cdot {\sum }_{n} δ ( t - n δ t - δ t / M ) = {\sum }_{n} s ( n δ t + δ t / M ) δ ( t - n δ t - δ t / M ) \qquad ( equation 2 )$$

for the second period of the signal; and

$$s ( t ) \cdot {\sum }_{n} δ ( t - n δ t - ( M - 1 ) δ t / M ) = {\sum }_{n} s ( n δ t + ( M - 1 ) δ t / M ) δ ( t - n δ t - ( M - 1 ) δ t / M ) \qquad ( equation 3 )$$

for the M period of the periodic signal. Where s(t) is the signal, δ(t) is a delta function used for representing the discrete (digital sampling), n is the number of data pieces used 5 for signal reconstructions, M is the number of periods of data collection and δt is the time difference between two consecutive data pieces of sampling in the rearranged signal.

This provides a sub sampling period shift of δt/M. As indicated above, the sub sampling period may be determined by pulse modulation of the illumination unit 200 rather than sampling rate and timing of the collection unit, thus enabling the use of relatively slow camera.

Further, the collected data pieces of the samples are rearranged in accordance with time location thereof with respect to the period of the measured signal. For example, first sample from the first period, first sample from the second period, first sample from the third period, second sample from the first period, second sample from the second period, second sample from the third period etc. The obtained result is a signal being sampled at 3 times the sampling frequency:

$$s ( t ) \cdot {\sum }_{n} δ ( t - n δ t / M ) = {\sum }_{n} s ( n δ t / M ) δ ( t - n δ t / M ) \qquad ( equation 4 )$$

where t refers to time and n is the number of data samples used for signal reconstruction.

Utilizing the example of speckle-based monitoring, the signal reconstruction may be based on determining a correlation between consecutive speckle patterns, i.e. sample data pieces. It should however be noted that the present technique may be used for other measurement systems where each measured data piece may or may not directly relates to the signal to be measured. To determine the correlation between the collected data pieces of the speckle patterns, the processing utility 600 may include a correlation module 630 configured for receiving arranged sequence of image data pieces from the arrangement module 620 and determine a correlation function between each pair of consecutive data pieces in the arranged set. The correlation function data is further transferred to the signal reconstruction module 640 for reconstruction of the signal. In embodiments where each sample relates directly to the measured signal, the arranged set of samples may be transmitted to the reconstruction module 640 for signal reconstruction.

As also indicated above, the control unit may include a stimulation module 650 connectable to the stimulation unit 400 and configured and operable to operate the stimulation unit 400 at a desired predetermined periodicity of stimulation. This allows measurement of response to external periodic signal, where the periodicity of the external force in known, rather than measuring internal periodic signal of the sample that may require providing data about the periodicity.

It should be noted that the processing of the sampled data may be done locally within the system 1000 or transferred for remote processing according to the above described technique. FIG. 3 illustrates the main operation of the technique of the invention in the form of a block diagram. As indicated above, to sample a periodic signal in (generally any) desired resolution, the technique includes providing data about periodicity of the periodic signal to be measured 3010. Based on the periodicity, the technique includes selecting and determining a sampling rate 3020 selected as described above to provide shifts of relative time of sampling instances with respect to different periods of the measured signal. The selected sampling rate may be fixed along a predetermined sampling time or utilize temporal shifts and delays between periods of the measured signal as described above. Sampling of the signal 3030 is performed using the suitable sampling technique in the selected sampling rate to provide a plurality of sampled data pieces for processing. Processing of the sampled data pieces includes arranging the data pieces in accordance with the corresponding temporal location within a period of the signal 3040 and reconstructing the signal 3050 from the arranged data.

Reference is made to FIGS. 4 and 5 exemplifying two techniques for sampling rate shifts that can be used with the technique of the invention. FIG. 4 shows a periodic signal s1(t) and a sampling train q1(t) having repetition rate selected such that there are non-integer number of sampling instances within each period of the signal s1(t). This provides shifts between time of sampling of different periodic of the signal s1(t). FIG. 5 shows sampling of another periodic signal s2(t) and sampling train q2(t). The sampling train is shifted after the first period τ by a selected time shift (determined in accordance with the desired reconstruction resolution) with respect to the location of the sampling instance of the first period q2′(t).

Thus, the technique of the invention provides for sampling a periodic signal at reduced sampling rate to thereby provide data enabling reconstruction of the signal profile at a desired resolution, greater than the sampling rate.

1. A method for use in detection of a periodic signal, the method comprising:
providing data about periodicity of the periodic signal;
sampling said periodic signal at a sampling rate along a plurality of periods of said signal, said sampling rate being selected as having temporal location of at least one sampling event along a corresponding period thereof varies for each period for a desired number of the periods;
arranging sampled data pieces in accordance with sample time thereof with respect to time within a period of said periodic signal; and
reconstructing a profile of said periodic signal from said plurality of periods being sampled at a desired resolution being greater than resolution associated with said sampling rate.

2. The method of claim 1, wherein said sampling rate is selected as having non-integer number of sampling events within a period of said periodic signal.

3. The method of claim 1, wherein said sampling events are temporally shifted between periods of said periodic signal.

4. The method of claim 1, wherein said sampling said periodic signals comprises providing pulsed illumination having said selected sampling rate at an inspection region, and collecting data about light returning from said inspection region at a predetermined frame-rate, integration time of light collection being longer than pulse duration of said pulsed illumination.

5. The method of claim 4, wherein said sampling rate of the pulsed illumination is determined such that each frame of the light collection corresponds to a single respective pulse of illumination.

6. A method for use in detection of periodic signal, the method comprising:
providing data about periodicity of the signal;
directing pulsed optical illumination of a selected repetition rate onto a region of interest for detection of said periodic signals;
collecting a plurality of data pieces corresponding to speckle patterns formed in light returning from said inspection region; and
processing said plurality of data pieces to determine a profile of said periodic signal;
wherein said processing comprises arranging said plurality of data pieces in accordance with temporal location of each data piece with respect to a period of said periodic signal and determining data about correlation between consecutive speckle patterns in the arranged order, said correlation data being indicative of profile of said periodic signal.

7. The method of claim 6, wherein said collection a plurality of data pieces comprises operating a collection unit at a predetermined frame rate and integration time being longer with respect to pulse duration of said pulsed optical illumination, thereby repetition rate and timing of said pulsed illumination determines a sampling of said signal.

8. The method of claim 6, wherein said repetition rate of the pulsed illumination is selected in accordance with said periodicity of the periodic signals such that illumination pulses are temporally shifted between periods of said periodic signal.

9. The method of claim 6, wherein said collection of a plurality of data pieces has a sampling rate corresponding to repetition rate of the pulsed illumination.

10. The method of claim 6, wherein said pulsed illumination is coherent illumination having a selected wavelength range.

11. The method of claim 6, wherein said providing data about the periodicity of the signal comprises stimulating the region of interest according to a predetermined periodic time profile, the periodicity of the signal being a dependent on the predetermined periodic time profile.

12. A system for use in monitoring of a signal, the system comprising:
an illumination unit configured for directing coherent pulsed illumination with a selected repetition rate to an inspection region;
a collection unit configured for collecting image data associated with speckle patterns generated by light returning from said inspection region; and
a control unit, the control unit is configured and operable for controlling operation of said illumination unit and said collection unit and for receiving from the collection unit input data corresponding with a plurality of image data pieces of said collected data about speckle patterns and for processing said input data for determining profile of one or more periodic signals;
wherein said control unit is configured for processing by arranging said plurality of image data pieces in accordance with temporal location of each data piece with respect to period of said periodic signal and determining data about correlation between consecutive speckle patterns in the arranged order.

13. The system of claim 12, wherein the control unit is configured and operable for operating the collection unit at a predetermined frame rate and integration time, and for synchronizing pulse timing of the illumination unit to provide sub-period sampling of said periodic signal.

14. The system of claim 12, wherein each frame captured by the light collection unit corresponds to a single respective pulse of illumination by the illumination unit.

15. The system of claim 12 wherein said control unit being configured for determining profile of said one or more periodic signal at a resolution greater than sampling rate of any of the illumination unit and collection unit.

16. The system of claim 12, wherein said collection unit comprises a camera unit configured for collecting defocused image data associated with light returning from the inspection region.

17. The system of claim 12, configured for sub-period sampling of said periodic signal, said control unit being configured for synchronizing pulse timing of said illumination unit to provide temporal shifts between illumination timing of different periods of said periodic signal.

18. The system of claim 17, wherein said control unit is configured for selecting pulse timing as having non-integer number of pulses within a period of said periodic signal.

19. The system of claim 17, wherein said control unit is configured for causing the illumination unit to emit a predetermined number of pulses during each period of said periodic signal and to temporally shift the predetermined number of signals between periods of said periodic signal.

20. The system of claim 12, further comprising a stimulation unit configured for stimulating the inspection region according to a periodic time profile.

21. The system of claim 20, wherein the control unit is configured for controlling an operation of said stimulation unit.
